SEQUENCE LISTING (1 sequence)

SEQ ID NO: 1 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

(19) United States
(12) Patent Application Publication
(10) Pub. No.: US 2022/0050118 A1
(43) Pub. Date: Feb. 17, 2022

(54) METHOD FOR DIAGNOSING A LIVER DISEASE

(71) Applicant: FIANOSTICS GmbH, Wiener Neustadt (AT)

(72) Inventors: Gerhard Hawa, Wien (AT); Albert Missbichler, Wien (AT)

(21) Appl. No.: 17/275,118

(22) PCT Filed: Sep. 11, 2019
(86) PCT No.: PCT/AT2019/060300

§ 371 Date: Mar. 10, 2021

(30) Foreign Application Priority Data
Sep. 12, 2018 (AT) A 50781/2018

(51) Int. Cl. G01N 33/68; G01N 33/543

(52) U.S. Cl. CPC G01N 33/6893; G01N 2800/085; G01N 33/543

(57) ABSTRACT

The present invention relates to a method for diagnosing a liver disease in a mammal comprising the step of determining the amount of a product encoded by the NOG gene in a biological fluid sample of said mammal and diagnosing a liver disease if the amount of the product encoded by the NOG gene in the sample of said mammal is different from the amount of the product encoded by the NOG gene determined in a sample of a healthy mammal.

### TECHNICAL FIELD

This invention relates to the detection of pathological changes in liver tissue by measuring a biomarker.

### BACKGROUND ART

Liver diseases like fatty liver disease (FLD) are very common pathology in the general population. It is noteworthy that in the Western population, malnutrition is the most common cause of non-alcoholic fatty liver disease (NAFLD), for instance, with an estimated incidence of 15 to 20%, and an increasing number of patients presenting risk factors for its development(Bedogni et al. 42(2005):44-52; Amarapurkar et al. Ann Hepatol 6(2007):161-163). Overnutrition- and obesity-related NAFLD is a multifactorial disorder and linked to hypertriglyceridemia, obesity, and insulin resistance, as observed in patients with metabolic syndrome (Higuchi and Gores, Curr Mol Med 3(2003):483-490).

Although FLD, for instance, is such a wide spread disease its noninvasive diagnosis remains an unmet medical need. Still the majority of patients have to undergo painful biopsy, since currently, this still is the gold standard for NAFLD diagnosis and staging. However, it is an invasive procedure and is limited by sampling error, high cost, procedure-related complications, and observer variability, even when performed by expert pathologists. Magnetic resonance imaging proton density fat fraction (MRI-PDFF) and magnetic resonance elastography (MRE) have emerged as accurate tools for quantifying steatosis but are very expensive and not accessible to all patients. They also have severe problems in detecting inflammation, which is a very important factor to estimate the progression of SS to NASH, which is key for patient stratification and therapy decisions. Therefore, there is a desperate need for non-invasive liver disease biomarkers, in particular NAFLD biomarkers, measured in body fluids like blood, to solve the above mentioned problems.

Thus, it is an object of the present invention to provide method and means allowing the diagnosis of liver diseases and the monitoring of the progress of liver diseases using non-invasive or minimal invasive methods.

### SUMMARY OF THE INVENTION

The present invention relates to a method for diagnosing a liver disease in a mammal comprising the step of determining the amount of a product encoded by the NOG gene in a biological fluid sample of said mammal and diagnosing a liver disease if the amount of the product encoded by the NOG gene in the sample of said mammal is different from the amount of the product encoded by the NOG gene determined in a sample of a healthy mammal of the same species.

It turned surprisingly out that the level of a product encoded by the NOG gene, preferably NOGGIN, in a biological fluid sample indicates whether a mammal from which said sample has been obtained suffers from a liver disease. One of the major advantages of the method of the present invention is the fact that the product encoded by the NOG gene can be measured in a biological fluid sample so that it is no longer necessary to perform a liver biopsy or any other invasive method in order to obtain a biological sample.

The present invention relates also to a non-invasive or minimal invasive method for diagnosing liver diseases like fatty liver diseases (FLD), in particular non-alcoholic fatty liver disease (NAFLD) or alcoholic fatty liver disease (AFLD).

The method of the present invention allows also discriminating between simple steatosis (SS) and nonalcoholic steatohepatitis (NASH). It has been found the amount of the product encoded by the NOG gene in the sample obtained from a mammal, in particular from a human, suffering from simple steatosis is significantly lower than in the sample from a mammal of the same species suffering from nonalcoholic steatohepatitis. The amount of the product encoded by the NOG gene in the sample obtained from a mammal suffering from simple steatosis is at least 20%, preferably at least 25%, lower compared to a sample from a mammal of the same species suffering from nonalcoholic steatohepatitis. Simple steatosis can be diagnosed in a mammal, in particular in a human, if the amount of the product encoded by the NOG gene in the sample is between 3 and 7 pmol/l, preferably between 4 and 6 pmol/l. Nonalcoholic steatohepatitis can be diagnosed in a mammal if the amount of the product encoded by the NOG gene in the sample is between 7.5 and 11 pmol/l, preferably between 8 and 10 pmol/l.

Another aspect of the present invention relates to a method for monitoring the progress of a liver disease or the treatment of a liver disease in a mammal comprising the step of determining the amount of a product encoded by the NOG gene in a biological fluid sample of said mammal.

Since the level of a product encoded by the NOG gene in a biological fluid sample of a mammal is influenced by the health status of the liver, the concentration of said NOG gene product can be directly used to monitor the progress of a liver disease or its treatment.

### BRIEF DESCRIPTION OF THE FIGURES

FIG. 1A shows serum noggin levels (mean±standard error of the mean) in patients with SS, NASH and controls. Serum noggin levels were much lower in SS and NASH patients than controls (p for trend=0.040), without being different between SS and NASH patients. *: p<0.05 compared to the control group

FIG. 1B shows serum log(noggin levels) (mean±standard error of the mean) in NAFLD patients randomly assigned to vitamin E monotherapy or to combined spironolactone and vitamin E therapy. Noggin levels increased similarly in both groups at month 2 and remained stable thereafter up to the end of the study. *: p<0.05 compared to the baseline noggin levels

### DESCRIPTION OF EMBODIMENTS

“Diagnosing” and “diagnosis”, as used herein, refer to methods by which a person skilled in the art can estimate and determine whether or not a mammal is suffering from a given disease or condition. This diagnosis is made on the basis of a biomarker, the amount (including presence or absence) of which is indicative of the presence, severity or absence of the condition.

“Liver disease”, as used herein, refers to any pathologic condition of the liver influencing its functioning.

“A product encoded by the NOG gene”, as used herein, refers to mRNA molecules, peptides, polypeptides, proteins and fragments thereof which are transcribed or translated from the coding region of the NOG gene.

The “NOG gene” codes for a protein called noggin (UniProtKB-Q13253) which is involved in the development of many body tissues, including nerve tissue, muscles and bones. Noggin is known to interact with members of a group of proteins called bone morphogenetic proteins (BMPs). BMPs help control the development of bone and other tissues.

Noggin is a secreted homodimeric glycoprotein that is an antagonist of bone morphogenetic proteins (BMPs). Human Noggin cDNA encodes a 232 amino acid (aa) precursor protein (UniProtKB-Q13253; SEQ ID No. 1); cleavage of a 27 aa signal peptide generates the 205 aa mature protein which contains an N-terminal acidic region, a central basic heparin-binding segment and a C-terminal cysteine-knot structure. So far NOGGIN has been under investigation in the area of dissemination of tumor cells to bone, ankylosing spondylitis or pulmonary arterial hypertension (PAH) but not with any pathology of the liver. Surprisingly the inventors found a strong association with a very common form of liver disease.

(UniProtKB - Q13253):
SEQ ID No. 1
MERCPSLGVTLYALVVVLGLRATPAGGQHYLHIRPAPSDNLPLVDLIEHP
DPIFDPKEKDLNETLLRSLLGGHYDPGFMATSPPEDRPGGGGGAAGGAED
LAELDQLLRQRPSGAMPSEIKGLEFSEGLAQGKKQRLSKKLRRKLQMWLW
SQTFCPVLYAWNDLGSRFWPRYVKVGSCFSKRSCSVPEGMVCKPSKSVHL
TVLRWRCQRRGGQRCGWIPIQYPIISECKCSC

“A sample of a healthy mammal”, as used herein, refers to a reference sample obtained by measuring the amount of a product encoded by the NOG gene in at least one, preferably at least two, more preferably at least five, more preferably at least ten, more preferably at least 20, mammals which do not suffer from any disease which is a result of or results in an unbalance of the noggin level including tumor, ankylosing spondylitis, pulmonary arterial hypertension (PAH), liver diseases and any other disease. “Healthy mammals” do not show any documented pathology of liver tissue. The sample of the healthy mammal is of the same source (e.g. blood, serum) and of the same origin (e.g. human, dog, cat, horse) as the biological fluid sample of the mammal which is examined in relation to liver diseases.

According to a preferred embodiment of the present invention a liver disease is diagnosed when the amount of the product encoded by the NOG gene in the sample of said mammal is significantly lower or higher, preferably at least 20%, preferably at least 25%, more preferably at least 30%, more preferably at least 40%, lower or higher, most preferably lower, compared to the amount of the product encoded by the NOG gene determined in a sample of a healthy mammal.

A liver disease is diagnosed if in a sample of a mammal the amount of the product encoded by the NOG gene is different from the amount of the product encoded by the NOG gene in a sample of a healthy mammal. It turned out that a difference of at least 25% indicates the presence of a liver disease.

According to another preferred embodiment of the present invention a liver disease is diagnosed when the amount of the product encoded by the NOG gene in the sample of said mammal, in particular human, is lower than 12 pmol/l, preferably lower than 11 pmol/l, more preferably lower than 10 pmol/l, more preferably lower than 9 pmol/l. The methods of the present invention allow to diagnose any liver disease or to monitor the treatment and/or progress of liver diseases. However, in a particularly preferred embodiment of the present invention the liver disease is a hepatic steatosis (fatty liver disease, FLD).

Hepatic steatosis (fatty liver) is characterized by an intracellular accumulation of lipids and subsequent formation of lipid droplets (LDl) in the cytoplasm of hepatocytes that is associated with an enlargement of the liver (hepatomegaly). When steatosis of the liver is further accompanied by inflammation, the condition is termed steatohepatitis. Both pathological conditions are subsumed under the term of nonalcoholic fatty liver disease (NAFLD) if alcohol can be excluded as a primary cause. Thus, NAFLD refers to steatosis as well to its progressive stages (i.e., steatohepatitis) Nonalcoholic fatty liver disease (NAFLD) includes simple steatosis (SS) and nonalcoholic steatohepatitis (NASH), which may advance to cirrhosis and hepatocellular carcinoma.

According to a preferred embodiment of the present invention the hepatic steatosis is selected from the group consisting of non-alcoholic fatty liver disease (NAFLD), preferably non-alcoholic steatohepatitis (NASH) or simple steatosis (SS).

According to another preferred embodiment of the present invention the product encoded by the NOG gene is Noggin (UniProtKB-Q13253).

Proteins, polypeptides and mRNA/cDNA encoding these molecules can be determined and/or quantified using methods well known in the art. According to a preferred embodiment of the present invention the amount of the product encoded by the NOG gene is determined by an immunoassay, ligand-receptor assay, protein microarray, mass spectroscopy method, biosensor or liquid chromatography method.

Particularly preferred are methods involving antibodies or fragments thereof capable to bind specifically products encoded by the NOG gene. Hence, the immunoassay is preferably selected from the group consisting of fluorescent immunoassay (FIA), enzyme-linked immunosorbent assay (ELISA) with chromogenic or luminometric detection and radioimmunoassay (RIA).

Particularly preferred immunoassays use fluorescence labelled antibodies. In order to enhance the sensitivity of such immunoassays these assays may be based on metal enhanced fluorescence as described, for instance, in WO 2017/046320.

According to a preferred embodiment of the present invention the biological fluid sample is a blood, serum, plasma, urine or salivary fluid sample.

According to another preferred embodiment of the present invention the mammal is a human subject, mouse, rat, bovine, equine, feline or canine subject.

Another aspect of the present invention relates to the use of a kit for determining the amount of a product encoded by the NOG gene in a biological fluid sample for diagnosing a liver disease in a mammal or for monitoring the progress of a liver disease or the treatment of a liver disease in a mammal.

Preferred kits may comprise antibodies or fragments thereof binding to the product encoded by the NOG gene, said antibodies or fragments thereof being optionally immobilized on a solid support, and fluorescently labelled antibodies or fragments thereof binding to the product encoded by the NOG gene.

In order to enhance the sensitivity of the detection method the solid support is preferably at least partially covered with a metal, preferably with silver. Particularly preferred solid supports are disclosed in WO 2017/046320.

The kit of the present invention may further comprise at least one calibrator containing specific amounts of Noggin protein, at least one control with a pre-defined amount of Noggin protein and/or at least one buffer for dilution of high reading samples, an enzyme or fluorophore labelled Noggin specific detection antibody preparation and a microplate coated with a Noggin specific capture antibody.

The microplate coated with a Noggin specific capture antibody comprises a structure surface and is at least partially covered with a metal coating as described in WO 2017/046320.

### EXAMPLES

The present invention is further illustrated by the following example, however, without being restricted thereto.

### Example

### Material & Methods

### Patients and Study Design

Inclusion criteria for NAFLD (“nonalcoholic fatty liver disease”) patients were: 1) age >18 years; 2) ultrasound imaging indicating fatty liver and abnormal liver function tests for at least 6 months before liver biopsy; and 3) patient's consent for liver biopsy. Age-sex-and body mass index (BMI)-matched individuals were recruited for control group, consisted of healthy individuals who underwent regular check-up for professional needs. Inclusion criteria for the controls were: 1) age >18 years; 2) no history of abnormal liver ultrasound imaging or abnormal liver function tests; 3) currently normal liver ultrasound imaging and normal liver function tests. Exclusion criteria were the same for patients and controls, targeting to exclude secondary causes of fatty liver, including medications or supplements possibly affecting NAFLD (Polyzos S, et al. Ann Hepatol. 2013;12(5):749-757).

The study was a one-center, 52-week, open label RCT (randomized controlled trial) with active control group. The RCT consisted of the screening visit, baseline visit, and three additional visits during the treatment phase (visit 2: week 8; visit 3: week 26; and visit 4: week 52).

Eligible NAFLD patients were randomized to receive per os vitamin E (400 IU/day in two equal doses; group 1) or spironolactone (25 mg once daily) plus vitamin E (400 IU/day in two equal doses; group 2) for 52 weeks. Randomization was performed with Excel (Microsoft Corp.) and allocation to treatment was done as described in Polyzos S A et al. (Diabetes Obes Metab. 2017;19(12):1805-1809).

### Analytic Methods

Anthropometric (weight, height, waist circumference) data were recorded and fasting morning (8-9 am) serum samples were collected in all visits. Laboratory tests for liver function (i.e. aspartate transaminase (AST), alanine transaminase (ALT), gamma-glutamyl transferase (GGT)) and glucose metabolism (i.e. glucose, insulin) were performed with standard methods using automated analyzers, as previously described (see Polyzos S A et al. Diabetes Obes Metab. 2017;19(12):1805-1809; and Polyzos S, et al. Ann Hepatol. 2013;12(5):749-757).

The serum concentration of noggin was measured using a high sensitive fluorescent immunoassay based on plasmonic microtiter plates (FluoBol™-Noggin; Fianostics GmbH, Austria), which increases the signal of fluorescent dyes several hundred-fold as described in Hawa G et al. (Anal Biochem. 2018 May 15;549:39-44). This assay detects free, bioactive human noggin, which is not bound to BMPs. Briefly, the assay protocol includes: adsorptive coating of capture antibody in 50 mM phosphate buffer (PBS)/150 mM NaCl pH 7.4, over-night at 4° C. followed by washing with PBS containing 0.1% Triton X-100. Blocking of unspecific binding was achieved with a proprietary solution of FIANOSTICS containing synthetic polymers and mercapto-compounds. After another washing step, 20 μl duplicates of standards/samples (serum) together with 25 μl of anti-human noggin antibody labelled with AlexaFluor680 were incubated over night at room temperature in the dark. Measurements were done using a standard fluorescence micro-plate reader. Samples reading above 100 pmol/l noggin were diluted with assay buffer and re-run to check for linearity of the signal. Inter-assay coefficient of variation (CV) was 2-7% and intra-assay CV 4-10%.

Liver biopsy was performed in all NAFLD patients under computed tomography-guidance and was interpreted according to the criteria of nonalcoholic steatohepatitis (NASH) Clinical Research Network (Kleiner D E, et al. Hepatology. 2005;41(6):1313-1321).

Body mass index (BMI), homeostasis model of assessment-IR (HOMA-IR), NAFLD liver fat score and AST-to-Platelet Ratio Index (APRI) were calculated, as previously described (see Polyzos S A et al. Diabetes Obes Metab. 2017;19(12):1805-1809). NAFLD liver fat score and APRI had been previously selected among four noninvasive indices of hepatic steatosis and five noninvasive indices of hepatic fibrosis, respectively, because they best fitted to the respective histological results of baseline, specifically for this RCT.

### Statistical Analysis

Continuous data are presented as mean±standard error of the mean (SEM). Kolmogorov-Smirnov test was used to check the normality of distributions of continuous variables. In case-control section, Chi-square or Fischer's exact test was used for comparisons between categorical variables. Spearman's coefficient (rs) was used for bivariate correlations. Independent samples T-test or Mann-Whitney test were used for comparisons between two groups of continuous variables. One-way analysis of variance (ANOVA) or Kruskal-Wallis test were used for comparisons of more than two groups of continuous variables. One-way analysis of co-variance (ANCOVA) was used to adjust for potential cofounders. Multiple linear regression analysis was used to investigate for independent associates of noggin.

In RCT section, two-way ANOVA was used to identify trends for differences within subjects, between subjects and within variable*time interaction, unadjusted or adjusted (two-way ANCOVA) for potential cofounders. The assumption of sphericity was tested with Mauchly's test of sphericity. Bonferroni correction was used, if needed, for multiple pairwise comparisons. Data of RCT were analysed using intention-to-treat analysis.

Variables that were not normally distributed were logarithmically transformed before entering in tests requiring the assumption of normal distributions. Significance was set at p<0.05 (two-tailed). Statistical analysis was performed with SPSS 21.0 for Macintosh (IBM Corp., Armonk, N.Y.).

### Results

### Case-Control Section

Thirty-one patients with histologically confirmed NAFLD (15 with SS, 16 with borderline or definite NASH) and 24 controls were included in this section. As specifically selected, there were not between group differences in sex, age, BMI and waist circumference. AST, ALT, GGT, glucose, insulin and HOMA-IR were statistically different between groups, with higher trends in NASH group.

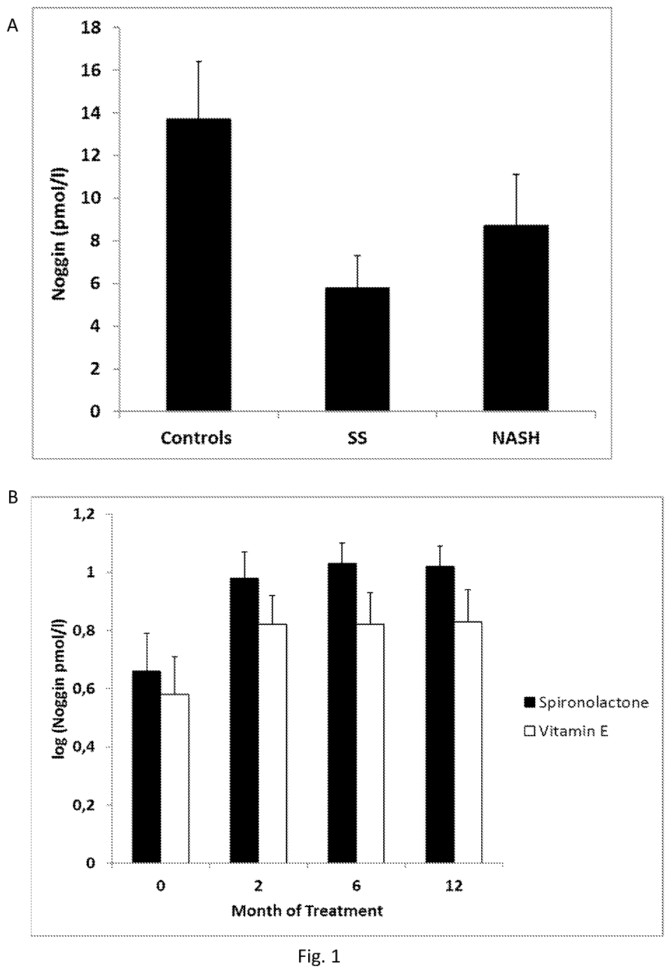

Noggin levels were lower in the entire NAFLD group (n=31; 7.4±1.5 pmol/l) than the control group (n=24; 13.7±2.7 pmol/l; p=0016). Similarly, noggin levels were lower in SS (5.8±1.5 pmol/l) and NASH (8.7±2.4 pmol/l) patients than the controls (13.7±2.7 pmol/l; p for trend=0.040) (see FIG. 1A). After sequential adjustment for age (model 1), age and sex (model 2), age, sex and log(ALT) (model 3), age, sex, log(ALT) and waist circumference (model 4), age, sex, log(ALT), waist circumference and log(HOMA-IR) (model 5), log(noggin) remained significantly different between groups (Table 1).

TABLE 1
Unadjusted and adjusted comparative data
between patients with SS, borderline and
definite NASH, and controls.

| | | | | p-value |
|---|---|---|---|---|
| | | | | for |
| | Controls | SS | NASH | trend* |
| Unadjusted | | | | |
| Log (noggin; | 0.96 ± | 0.55 ± | 0.68 ± | 0.028 |
| pmol/l) | 0.09 | 0.12 ^{a} | 0.13 | |
| Model 1 | | | | |
| Log (noggin; | 0.96 ± | 0.55 ± | 0.68 ± | 0.030 |
| pmol/l) | 0.10 | 0.13 ^{a} | 0.12 | |
| Model 2 | | | | |
| Log (noggin; | 0.95 ± | 0.55 ± | 0.68 ± | 0.039 |
| pmol/l) | 0.10 | 0.13 ^{a} | 0.12 | |
| Model 3 | | | | |
| Log (noggin; | 1.06 ± | 0.51 ± | 0.55 ± | 0.015 |
| pmol/l) | 0.12 | 0.13 ^{a} | 0.14 | |
| Model 4 | | | | |
| Log (noggin; | 1.02 ± | 0.50 ± | 0.70 ± | 0.020 |
| pmol/l) | 0.11 | 0.13 ^{a} | 0.15 | |
| Model 5 | | | | |
| Log (noggin; | 0.99 ± | 0.52 ± | 0.69 ± | 0.046 |
| pmol/l) | 0.11 | 0.13 ^{a} | 0.16 | |

Data are presented as mean ± standard error of the mean (SEM) for unadjusted values and as estimated marginal mean ± standard error of the mean (SEM) for adjusted values.
^{a} p < 0.05 compared to the control group (Bonferroni post-hoc adjustment)
Model 1: adjustment for age;
model 2: adjustment for age and sex;
model 3: adjustment for age, sex and log (ALT);
model 4: adjustment for age, sex, log (ALT) and waist circumference;
model 5: adjustment for age, sex, log (ALT), waist circumference and log (HOMA-IR).
Abbreviations:
ALT, alanine transaminase;
HOMA-IR, homeostatic model assessment insulin resistance;
NASH, nonalcoholic steatohepatitis;
SS, simple steatosis;.

Within patients (n=31), noggin levels were not different between groups of different grade of steatosis, portal and lobular inflammation, ballooning, and fibrosis.

### RCT Section

Thirty-one NAFLD patients (15 with SS and 16 with NASH) were randomly assigned to group 1 (n=17; 11 women) or group 2 (n=14; 12 women). At baseline, the two groups were similar for all parameters and there were no differences in adverse events during treatment.

Log(noggin) levels similarly increased after treatment in both groups (group 1; baseline: 0.66±0.13; month 2: 0.98±0.09; month 6: 1.03±0.07; month 12: 1.02±0.07 pmol/l, and group 2; baseline 0.58±0.13; month 2: 0.82±0.10; month 6: 0.82±0.11; month 12: 0.83±0.11 pmol/l; FIG. 1B). More specifically, log(noggin) was not different between groups (p=0.20), but increased within groups over time (p<0.001). There was not significant difference in the group*time interaction (p=0.62). After correction for multiple comparisons, log(noggin) significantly increased at month 2 (p=0.008 compared to baseline) and remained stable at month 6 (p=0.005 compared to baseline) and 12 (p=0.001 compared to baseline) without further increasing (see FIG. 1B).

### Discussion

Lower noggin levels were shown for the first time in NAFLD (SS and NASH). Noggin levels increased similarly after a 2-month treatment with vitamin E monotherapy or the combination of spironolactone and vitamin E, presumably owing to vitamin E action.

Since the pathogenesis of NAFLD is multifactorial, a combination treatments rather than monotherapy may be more effective by simultaneously targeting more than one pathogenic factors. However, the addition of spironolactone to vitamin E did not further increase noggin. Although noggin is increased by vitamin E, its change was not associated with changes in indices of hepatic steatosis and indices, implying that it does not affect them.

In conclusion, lower noggin levels were observed in NAFLD patients than controls, and noggin levels increased similarly after combined low-dose spironolactone plus vitamin E or vitamin E monotherapy in NAFLD patients.

1. A method for diagnosing a liver disease in a mammal comprising the step of determining the amount of a product encoded by the NOG gene in a biological fluid sample of said mammal and diagnosing a liver disease if the amount of the product encoded by the NOG gene in the sample of said mammal is different from the amount of the product encoded by the NOG gene determined in a sample of a healthy mammal.

2. The method according to claim 1, wherein a liver disease is diagnosed when the amount of the product encoded by the NOG gene in the sample of said mammal is significantly lower or higher, compared to the amount of the product encoded by the NOG gene determined in a sample of a healthy mammal.

3. The method according to claim 1, wherein a liver disease is diagnosed when the amount of the product encoded by the NOG gene in a mammalian sample, is lower than 12 pmol/l.

4. A method for monitoring the progress of a liver disease or the treatment of a liver disease in a mammal comprising the step of determining the amount of a product encoded by the NOG gene in a biological fluid sample of said mammal.

5. The method according to claim 1, wherein the liver disease is a hepatic steatosis (fatty liver disease, FLD).

6. The method according to claim 5, wherein the hepatic steatosis is selected from the group consisting of non-alcoholic fatty liver disease (NAFLD), preferably non-alcoholic steatohepatitis (NASH) or simple steatosis (SS).

7. The method according to claim 1, wherein the product encoded by the NOG gene is Noggin.

8. The method according to claim 1, wherein the amount of the product encoded by the NOG gene is determined by an immunoassay, ligand-receptor assay, protein microarray, mass spectroscopy method, biosensor or liquid chromatography method.

9. The method according to claim 8, wherein the immunoassay is selected from the group consisting of fluorescent immunoassay (FIA), enzyme-linked immunosorbent assay (ELISA) with chromogenic or luminometric detection and radioimmunoassay (RIA).

10. The method according to claim 1, wherein the biological fluid sample is a blood, serum, plasma, urine or salivary fluid sample.

11. The method according to claim 1, wherein the mammal is a human subject, mouse, rat, bovine, equine, feline, or canine subject.

12. Use of a kit for determining the amount of a product encoded by the NOG gene in a biological fluid sample for diagnosing a liver disease in a mammal or for monitoring the progress of a liver disease or the treatment of a liver disease in a mammal.

13. Use according to claim 12, wherein the kit comprises antibodies or fragments thereof binding to the product encoded by the NOG gene, said antibodies or fragments thereof being optionally immobilized on a solid support, and fluorescently labelled antibodies or fragments thereof binding to the product encoded by the NOG gene.

14. Use according to claim 13, wherein the solid support is at least partially covered with a metal, preferably with silver.

15. Use according to claim 12, wherein the kit comprises further at least one calibrator containing specific amounts of Noggin protein, at least one control with a pre-defined amount of Noggin protein and/or at least one buffer for dilution of high reading samples, an enzyme or fluorophore labelled Noggin specific detection antibody preparation and a microplate coated with a Noggin specific capture antibody.

16. Use according to claim 12, wherein the microplate coated with a Noggin specific capture antibody comprises a structure surface and is at least partially covered with a metal coating.
